(19) United States
(12) Patent Application Publication
(10) Pub. No.: US 2021/0196903 A1
(43) Pub. Date: Jul. 1, 2021

(54) CATHETER TEST APPARATUS, KITS, AND METHODS

(71) Applicant: Merit Medical Systems, Inc., South Jordan, UT (US)

(72) Inventors: Fred Lampropoulos, Salt Lake City, UT (US); Steven Weir, Salt Lake City, UT (US); Gregory R. McArthur, Sandy, UT (US)

(21) Appl. No.: 17/131,310

(22) Filed: Dec. 22, 2020

Related U.S. Application Data
(60) Provisional application No. 62/953,839, filed on Dec. 26, 2019.

(51) Int. Cl. A61M 5/50; A61M 5/32; A61M 5/315

(52) U.S. Cl. CPC A61M 5/5086; A61M 2205/584; A61M 5/31501; A61M 5/3202

(57) ABSTRACT

Apparatus, methods, and kits are for testing a catheter for an obstruction with a lumen by a medical practitioner at the point of use. In some embodiments, an elongate member, including elongate members comprising a gauge portion, is inserted into the lumen configured to detect a reduced diameter condition.

### RELATED APPLICATIONS

This application claims priority to U.S. Provisional Application No. 62/953,839, filed on Dec. 26, 2019 and titled, “Catheter Test Apparatus, Kits, and Methods,” which is hereby incorporated by reference in its entirety.

### TECHNICAL FIELD

The present disclosure relates generally to the field of medical devices. More specifically, in some embodiments, the present disclosure relates to a test apparatus to verify or assess the size of lumen. Related methods are also disclosed.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a perspective view of catheter test apparatus according to a first embodiment.

FIG. 2 is an exploded side view of the catheter test apparatus of FIG. 1.

FIG. 3A is an illustration of the catheter test apparatus of FIG. 1 in testing engagement with a catheter.

FIG. 3B is a detail view of a sectioned portion of the catheter showing a blockage and a distal end portion of the elongate member within the catheter lumen.

FIG. 4A is an illustration of the catheter test apparatus of FIG. 1 in the failure detected state.

FIG. 4B is a detail view of the sectioned portion of the catheter showing the distal end of the elongate member abutting the blockage.

FIG. 5 is a side view of catheter test apparatus according to a second embodiment.¶

FIG. 6 is an illustration of the catheter test apparatus of FIG. 5 in testing engagement with a catheter.

FIG. 7 is an illustration of the catheter test apparatus of FIG. 5 in the failure detected state.

FIG. 8 is a side view of catheter test apparatus according to a third embodiment.

FIG. 9 is an illustration of the catheter test apparatus of FIG. 8 in testing engagement with a catheter.

FIG. 10 is an illustration of the catheter test apparatus of FIG. 8 in the failure detected state.

FIG. 11A is a perspective view of catheter test apparatus according to a fourth embodiment.

FIG. 11B is a cross-sectional view of a clamp of the catheter test apparatus of FIG. 11A.

FIG. 12 is a perspective view of kit containing a catheter test apparatus and a vascular device.

### DETAILED DESCRIPTION OF PREFERRED EMBODIMENTS

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure but is merely representative of various embodiments. While the various aspects of the embodiments are presented in drawings, the drawings are not necessarily drawn to scale unless specifically indicated.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest the practitioner during use.

“Fluid” is used in its broadest sense, to refer to any fluid, including both liquids and gases as well as solutions, compounds, suspensions, etc., which generally behave as fluids.

Vascular therapies and procedures may include concentric vascular devices or one vascular device disposed within another vascular device. Vascular devices such as catheters may be configured to be disposed in small vasculature pathways. In some therapies, minimizing or limiting the combined catheter size or profile may facilitate treatment of small vessels, tortuous anatomies, and so forth. Thus, concentric catheters may be configured such that, when one catheter is disposed within another catheter, there may be very little clearance between the two catheters. Thus, deviations from a nominal size (such as those due to manufacturing tolerances) may prevent concentric catheters from properly operating. For example, if the inside diameter of the outer catheter is too small, a practitioner may be unable to advance the desired inner catheter within the outer catheters. With regard deviations from nominal due to manufacturing processes, in some instances manufacturing processes associated with the end points of a catheter tube may be more prone to causing manufacturing defects such as undersized lumens. As such, the end points of a catheter may comprise defects more often than the tubing extending between the end points. Again, in some instances, defects causing an internal diameter of the lumen of the catheter to be less than a specified tolerance limit may prevent the insertion of another catheter within the lumen.

During some therapies, a defect of an outer catheter preventing use with a particular inner catheter may not be discovered until an attempt is made to insert the inner catheter into the outer catheter. At that point, the outer catheter may already be inserted into the vasculature of a patient and the inner catheter may already be removed from its sterile package. In such a case, both catheters may no longer be in a condition for use. As such, a non-defective catheter may need to be discarded as a result of a defect of the other catheter. This may further require repositioning of a new outer catheter which may increase treatment time. It may also be the case that, if an inner catheter will not fit within the lumen an outer catheter, the cause may not be readily apparent, i.e., it may not be apparent which catheter is defective. If such an occurrence does take place, it may be detrimental to the patient and it may result in significant cost to the care provider. A device for assessing the defect of an outer catheter before opening a sterile package of the inner catheter may result in savings to the provider and reduced risk to a patient.

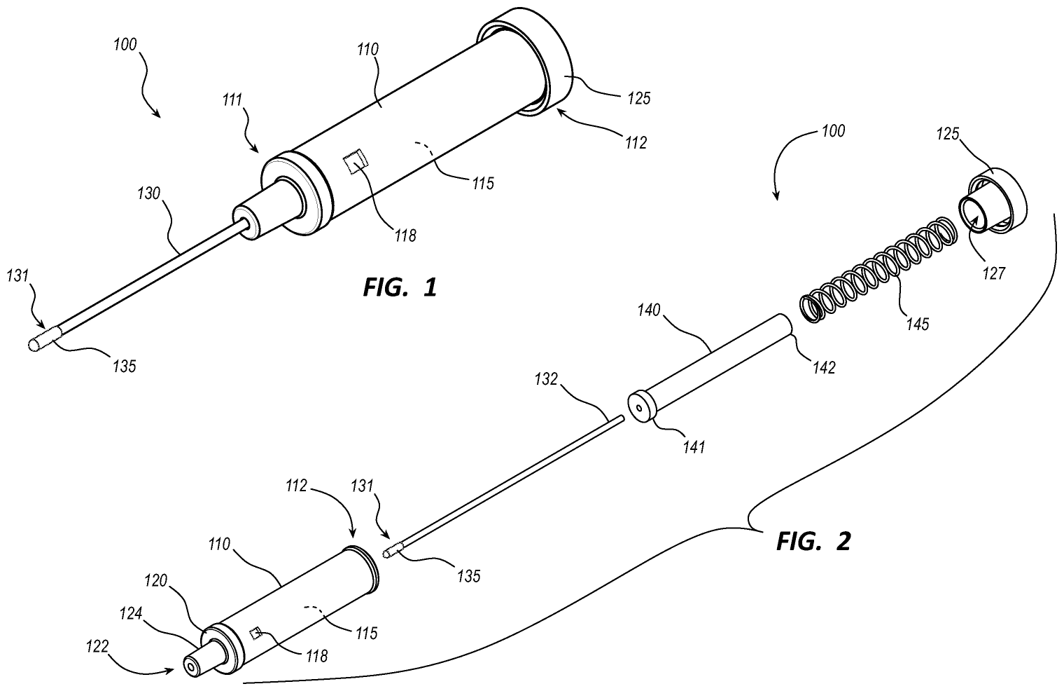

FIG. 1 is a perspective view of a catheter test apparatus 100, and FIG. 2 is an exploded assembly view of the catheter test apparatus 100. The catheter test apparatus 100 may include the following components: a body 110, an elongate member 130, a piston 140, and a biasing member 145. Further detail of each component and their structural and functional interrelationships follows below. The elongate member 130 (such as at a proximal end 132 of the elongate member 130) is coupled to the piston 140 so as to extend distally away from a distal end 141 of the piston 140. The piston 140 and the elongate member 130 are disposed within a chamber 115 of the body 110 such that the elongate member 130 extends distally through an aperture 122, the aperture 122 extending through a distal end cap 120 of the body 110. The elongate member 130 extends distally further away from a distal end 111 of the body 110. The chamber 115 may be sized to provide for longitudinal displacement of the piston 140 through at least a portion of the chamber 115, which displacement may be slidable. The piston 140 may be shaped to interface with the biasing member 145 so that the biasing member 145 exerts a force on the piston 140 in a distal direction. In some embodiments, such as the embodiment shown in FIG. 2, the biasing member 145 may be a coil spring. Other types of springs, resilient materials, and compliant mechanisms may be used to form the biasing member. The body 110 may comprise a proximal end cap 125 which may in some embodiments comprise an aperture 127 sized to receive at least portion of the piston 140 therethrough. Either of the distal end cap 120 and the proximal end cap 125 may be integrally formed with the body 110. The distal end cap 120 may comprise a distal tip portion 124 extending distally away from the distal end 111 of the body 110. The distal tip portion 124 may be sized to interface with a catheter hub as further described below.

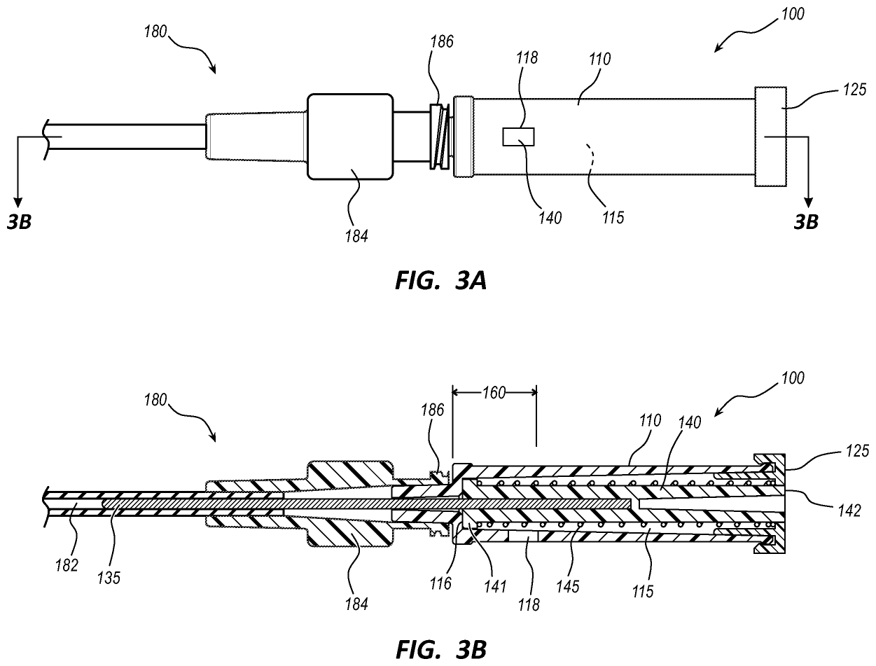

In the illustrated embodiment, the elongate member 130, coupled to the piston 140, is urged in a distal direction by the biasing member 145 such that an external force applied to the elongate member 130 in the proximal direction of sufficient magnitude may proximally displace the elongate member 130 and the piston 140 proximally within the chamber 115 in opposition to the biasing member 145. The biasing member 145 may be sized to exert a preload force of a predetermined magnitude on the piston 140 when the distal end 141 of the piston 140 is disposed adjacent a distal end 116 (see FIG. 3B) of the chamber 115. The piston 140 may be sized to be disposed completely within the chamber 115 when the distal end 141 of the piston 140 is disposed adjacent the distal end 116. The piston 140 may also be sized such that a proximal end 142 of the piston 140 is visible to a practitioner when the piston 140 is displaced away from the distal end 116. The body 110 may comprise an opening 118 disposed on the side of the body 110 extending through a wall of the body 110 to the chamber 115 as further described below.

The elongate member 130 may be rigid. In some embodiments, the elongate member 130 may comprise rigid portions and flexible portions. In some embodiments, the elongate member 130 may include a gauge portion 135 disposed at a distal end 131 of the elongate member 130. The gauge portion 135 may be cylindrical in shape and may comprise a specified nominal diameter. The gauge portion 135 may be manufactured such that the diameter is within a specified range or tolerance. The nominal diameter may be between about 0.03 and 0.1 inches, 0.03 and 0.08 inches, 0.03 and 0.05 inches, 0.035 and 0.045 inches, or 0.039 and 0.041 inches. The specified tolerance may be plus/minus 0.0005 inches, 0.0002 inches, 0.0001 inches, or less. Embodiments wherein the entire elongate member 130 is a constant diameter (thus there is no difference along the length of the elongate member 130 in the diameter of the elongate member 130 and the gauge portion 135) are likewise within the scope of this disclosure.

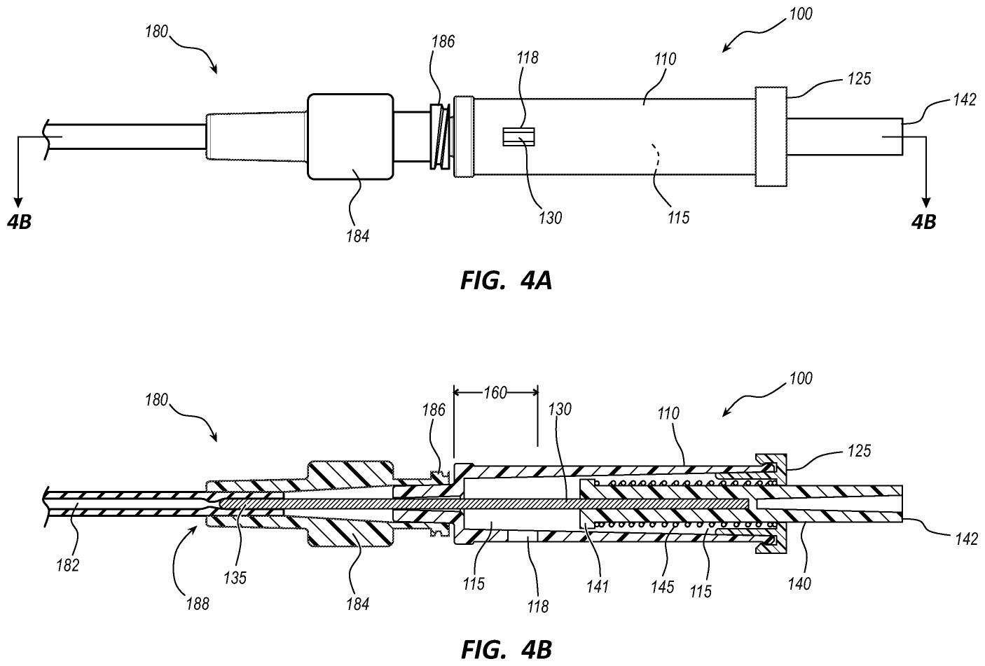

The catheter test apparatus 100 may be configured to indicate compatibility of a vascular device with a catheter. More specifically, the catheter test apparatus 100 may indicate if a vascular device may be inserted within a lumen of the catheter. In some instances, the catheter test apparatus 100 may provide indication related to presence or absence of an obstruction within the lumen of the catheter. An obstruction may be any condition of a catheter lumen that may interfere with or otherwise affect the insertion of a vascular device. In other words, the catheter test apparatus 100 may indicate if a portion of the lumen of the catheter comprises a minimum diameter that is less than a maximum outside diameter of the vascular device thus preventing insertion of the vascular device within the lumen of the catheter. FIGS. 3A, 3B, 4A and 4B illustrate use of the catheter test apparatus 100.

FIG. 3A is a side view illustration of the catheter test apparatus 100 in operative engagement with a catheter 180, i.e., a catheter to be tested for obstruction. FIG. 3B is a cross sectional side view of the catheter test apparatus 100 and the catheter 180 of FIG. 3A cut along sectioning lines 3B-3B. During certain uses, the elongate member 130 is inserted into a lumen 182, and the body 110 is displaced toward a catheter hub 184 such that the distal end 111 of the body 110 is disposed adjacent the proximal end of the catheter hub 184. The gauge portion 135 of the elongate member 130 is inserted within the lumen 182 of the catheter 180 extending distally beyond the distal end of the catheter hub 184. FIGS. 3A and 3B illustrate a condition of the catheter 180 absent an obstruction. In other words, FIGS. 3A and 3B illustrate a condition wherein the catheter test apparatus 100 is providing an indication to the medical practitioner of the absence of an obstruction. For example, as the gauge portion 135 is advanced into a catheter 180 lumen, if the force required to advance the gauge portion 135 is less than the force required to compress the biasing member 145 and displace the piston 140, the catheter test apparatus 100 will be in the position shown in FIGS. 3A and 3B as the elongate member 130 is advanced within the catheter 180.

The catheter 180, may be understood as representative of any catheter—or any device with an inside diameter—to be tested, and as stated above comprises the lumen 182. The catheter 180 may include a specified diameter range for the lumen 182, which specified diameter range may include a minimum specified diameter. Again, FIGS. 3A and 3B illustrate a condition of the catheter 180 where the diameter of the lumen 182 is greater than the diameter of the gauge portion 135. In some embodiments, the gauge portion 135 may be sized to be equal to the minimum specified diameter of the lumen 182, and therefore, the catheter test apparatus 100 may indicate that the diameter of the lumen 182 is greater than its minimum specified diameter or that there is no obstruction causing the minimum diameter of the lumen 182 to be less than its minimum specified diameter.

The catheter 180 may include a catheter hub 184. The catheter hub 184 may include external threads 186. In some embodiments, the catheter hub 184 may comprise a female Luer hub and as such may in some respects comply with the ISO 80369-7 standard. In other embodiments, the catheter hub 184 may comprise a male Luer connector. In some embodiments, the distal tip portion 124 may be configured to interface with a female Luer connector or a male Luer connector. As such, the distal tip portion 124 may in some respects comply with the ISO 80369-7 standard.

Similar to FIG. 3A, FIG. 4A is also a side view illustration of the catheter test apparatus 100 in operative engagement with the catheter 180, and FIG. 4B is a similar cross-sectional side view of the catheter test apparatus 100 and the catheter 180 of FIG. 4A cut along sectioning lines 4B-4B. In the configuration shown in FIGS. 4A and 4B, the elongate member 130 is inserted into the lumen 182, and the body 110 is displaced toward the catheter hub 184 such that the distal end 111 of the body 110 is disposed adjacent the proximal end of the catheter hub 184. The gauge portion 135 of the elongate member 130 is inserted within the lumen 182 of the catheter 180 extending distally within the lumen 182 and engaging an obstruction 188 such that further insertion of the elongate member 130 in inhibited. As such, FIGS. 4A and 4B illustrate a condition where the catheter test apparatus 100 is providing a visual indication to the medical practitioner of the presence of the obstruction 188 within the lumen 182.

In this illustrated instance, the obstruction 188 applies a proximally directed reaction force on the elongate member 130 resulting in proximal displacement of the piston 140 with respect to the body 110 in opposition to the biasing member 145. The piston 140 is positioned proximal of the predetermined distance 160. As such, the location of the piston 140 provides indication of the presence of the obstruction 188. As shown in FIGS. 4A and 4B, the proximal end 142 of the piston 140 may extend proximally beyond a proximal end 112 of the body 110 providing a visible indication of the position of the piston 140.

Stated another way, when the force required to advance the gauge portion 135 is sufficient to compress the biasing member 145, the piston 140 may extend from the body 110 to indicate to the practitioner that the catheter lumen 182 may be too small to advance an inner catheter. The gauge portion 135 may be sized to correlate to the diameter, or may be sized just larger than, the size of an inner catheter. Thus, displacement of the piston 140 as the gauge portion 135 is advanced into the catheter lumen 182 may indicate that the catheter is not compatible for use with an inner catheter that correlates with the catheter test apparatus 100. Similarly, if the force required to advance the gauge portion 135 is less than the force required to compress the biasing member 145, the piston 140 will not be displaced with respect to the body 110. This condition may be understood as an indication that the catheter 180 is properly sized for use with an inner catheter that correlates to the catheter test apparatus 100.

The size of the elongate member 130, including the gauge portion 135, may be slightly larger than the nominal or known size of the outside diameter of a correlating inner catheter. Further, the biasing member 145 may be selected to provide a particular force, depending on the desired characteristics of the catheter test apparatus 100. Lower biasing forces and/or larger gauge portions 135 may increase the degree of confidence that an inner catheter will indeed fit, though these characteristics could also result in outer catheters displacing the piston 140 (as shown in FIG. 4A) even when an inner catheter would actually fit. Thus, these parameters can be adjusted or selected during manufacturing of the catheter test apparatus 100 depending on the desired confidence interval of the test.

The body 110 may comprise an opening 118 in a side wall to provide for visible observation of the piston 140 within the chamber 115. In some embodiments, the opening 118 may be positioned such that the piston 140 is visible through the opening 118 when the catheter test apparatus 100 detects the absence of an obstruction 188 in the lumen 182 and not visible through the opening 118 when the catheter test apparatus 100 detects the presence of an obstruction 188. Alternatively, the opening 118 may be positioned such that the piston 140 is not visible through the opening 118 when the catheter test apparatus 100 detects the absence of an obstruction 188 in the lumen 182 and visible through the opening 118 when the catheter test apparatus 100 detects the presence of an obstruction 188. In other embodiments, other types of visual observance may indicate the presence or absence of an obstruction 188, such as differing colored portions of the plunger 140. In other embodiments, the body 110 may comprise two or more openings 118 such that visual observance of the plunger 140 through one opening may indicate detection of an obstruction 188, and visual observance of the plunger 140 through another opening may indicate detection of the absence of an obstruction 188. One of ordinary skill will recognize other ways of providing visual indication of the location of plunger 140 within the chamber 115 all of which are included within this disclosure. In some embodiments, the body 110 may be formed of a transparent or translucent material such that the medical practitioner can visually observe the piston 140 or more specifically the position of the piston 140 within the body 110. As the elongate member 130 is coupled to the piston 140 at the proximal end 132, the position of the piston 140 may correlate with the position of the proximal end 132 of the elongate member 130.

The length of the elongate member 130 may be defined by an anticipated location of the obstruction 188 along the length of the lumen 182. For instance, due to manufacturing techniques, the end portions of the catheter 180 may more commonly contain defects, such as obstructions or narrow portions, than the portion of the lumen 182 extending between the end portions. Thus, testing of just a portion of an end (such as the portion within the catheter hub 184) may be sufficient to provide a desired confidence level that the catheter 180 will work with a particular inner catheter. It may also be the case that quality data or experience associated with a particular type or design of catheter 180 that may indicate that a proximal end portion of the catheter has an increased propensity for an obstruction 188. For example, the attachment of the catheter hub 184 may be the cause for an obstruction 188, and therefore, insertion of the gauge portion 135 beyond the distal end of the catheter hub 184 may provide sufficient assurance desired by the medical practitioner. Stated another way, the catheter test apparatus 100 may be configured to only test a portion of the catheter 180 adjacent an end of the catheter 180. As such, the length of the elongate member 130 may be sized at least to extend from the distal end of the catheter hub 184 to the distal end 141 of the piston 140 when the piston 140 adjacent the distal end 116 of the chamber 115 and when the distal end 111 of the body 110 is positioned adjacent the proximal end of the catheter hub 184. As such, the predetermined distance 160 may at least partially be defined by the length of the elongate member 130. Again, in certain instances, a test result indicating that the catheter 180 is obstruction free if the proximal end portion does not contain an obstruction 188 may be sufficient for a practitioner to decide to proceed with a procedure (even understanding that there is a possibility of an obstruction at some point further down the length of the catheter 180).

The biasing member 145 may be constructed to provide a force on the piston 140. In some embodiments, the magnitude of the force may be at least partially defined by a typical frictional force opposing insertion of the vascular device within the lumen 182 of the catheter 180. A magnitude of force provided by the biasing member 145 that is marginally greater than the typical frictional force will provide a greater level of obstruction detection sensitivity than a magnitude which is significantly greater than the typical frictional force. In some embodiments, the magnitude of the force may be at least partially defined by a vascular device's capability to accommodate a compressive force without buckling during insertion. In some embodiments, the magnitude of force provided by the biasing member 145 may be between about 30 and 300 grams, 30 and 100 grams, 40 and 60 grams, or 45 and 55 grams.

Use of the catheter test apparatus 100 described above to test a catheter for the presence of an obstruction may include any of the following steps or processes, each of which may be optional or interchanged. The following steps or processes may be performed in any order unless specifically defined. The following steps or processes may also be performed at a location other than the location of manufacture of the catheter. The following steps or processes may also be performed at the location of use of the catheter. The following steps or processes may also be done while the catheter is inserted within the vasculature of a patient.

The medical practitioner may provide a catheter to be used in a vascular procedure which may include removing the catheter from a sterile package. The medical practitioner may provide a catheter test apparatus which may include removing the catheter from a sterile package. Providing the catheter test apparatus may include separating the sterile package containing the catheter test apparatus from a sterile package containing a vascular device to be inserted into the catheter to be tested without compromising the sterility of the vascular device and/or the catheter test apparatus. The medical practitioner may determine the suitability of the catheter test apparatus to test the catheter by comparing information contained in indicia disposed on the sterile package of the catheter test apparatus with one or more parameters of the catheter or vascular device.

The medical practitioner may insert the elongate member including the gauge portion at the distal end of the elongate member through the hub so as to be adjacent the proximal opening of a lumen of the catheter. The medical practitioner may displace the body toward the hub of the catheter causing the elongate member to extend into the lumen of the catheter until the distal end of the body is disposed adjacent the proximal end of the catheter hub. The medical practitioner may visually observe the location of the proximal end of the elongate member which may corresponded to the location of the piston and determine if the location of the proximal end of the elongate member and/or the piston is distal or proximal of the predetermined distance from the hub. The medical practitioner may: 1) conclude that the catheter is obstruction free if the proximal end of the elongate member and/or the piston is distal of the predetermined distance or 2) conclude that the catheter contains an obstruction if the proximal end of the elongate member and/or the piston is proximal of the predetermined distance. In some embodiments, the medical practitioner may observe the proximal end 112 of the body 110, to see if a portion of the piston 140 is extending beyond the proximal end 112 which may correlate to a position of the piston 140 proximal of the predetermined distance 160.

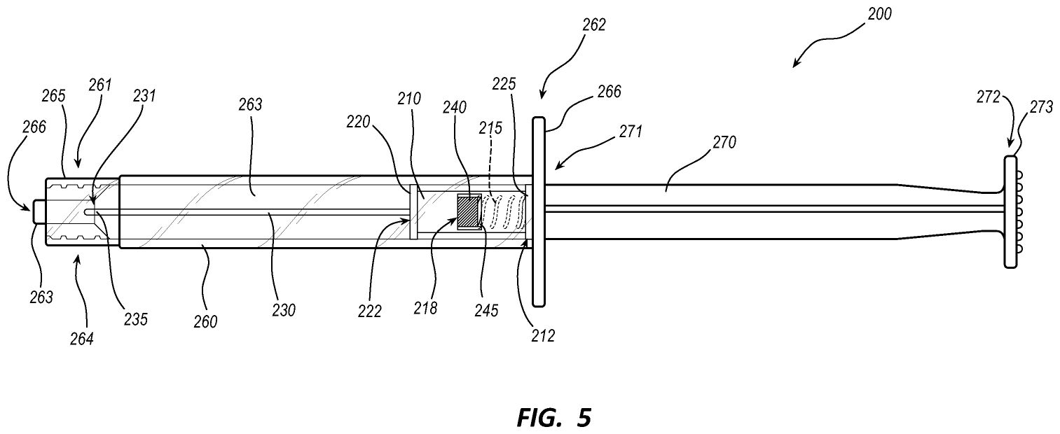

FIGS. 5, 6, and 7 illustrate another embodiment of a catheter test apparatus 200 that can, in certain respects, resemble components of the catheter test apparatus 100 described in connection with FIGS. 1, 2, 3A, 3B, 4A, and 4B. It will be appreciated that the illustrated embodiments may have analogous features. Accordingly, like features are designated with like reference numerals, with the leading digits incremented to “2.” For instance, the body is designated as “110” in FIGS. 1, 2, 3A, 3B, 4A, and 4B, and a body is designated as “210” in FIGS. 5, 6 and 7. Relevant disclosure set forth above regarding similarly identified features thus may not be repeated hereafter. Moreover, specific features of the catheter test apparatus 100 and related components shown in FIGS. 1, 2, 3A, 3B, 4A, and 4B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the system of catheter test apparatus 200 of FIGS. 5, 6, and 7. Any suitable combination of the features, and variations of the same, described with respect to the system of the catheter test apparatus 100 and components illustrated in FIGS. 1, 2, 3A, 3B, 4A, and 4B can be employed with the system of the catheter test apparatus 200 and components of FIGS. 5, 6, and 7 and vice versa.

Referring to FIG. 5, the catheter test apparatus 200 may comprise components of the catheter test apparatus 100 and further comprise a housing 260 and a plunger rod 270 as further described below. The housing 260 includes a cavity 263 extending longitudinally between a distal end cap 265 at a distal end 261 and an open proximal end 262. The cavity 263 is sized for disposition of the body 210 and at least a portion of an elongate member 230 therein. In some embodiments, the cavity 263 may be sized for disposition of a substantial entirety the elongate member 230 therein. The housing 260 may be transparent or translucent such that the presence and position of the body 210 therein are visible to the medical practitioner. The housing 260 may also include one or more finger flanges 266 disposed adjacent the proximal end 262 so as to provide for the application of a proximally directed force on the housing 260 by one or more fingers of the medical practitioner.

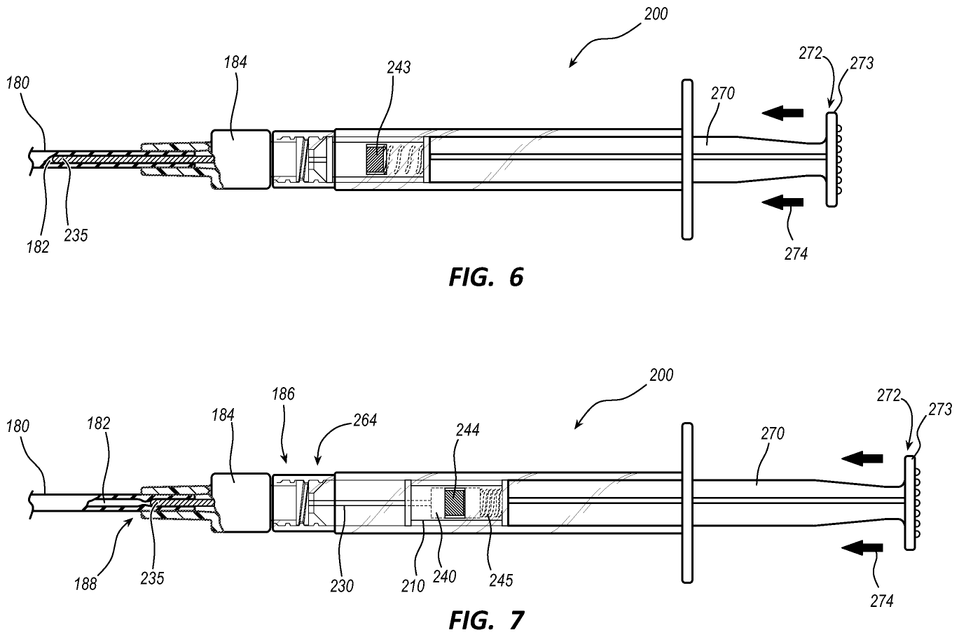

The distal end cap 265 includes an aperture 266 configured for passage of the elongate member 230 therethrough. The distal end cap 265 may also include a distal tip portion 263, and the aperture 266 may also extend through the distal tip portion 263. The distal end cap 265 may include threads 264 configured to engage threads of the catheter hub 184 (see FIGS. 6 and 7). The distal end cap 265, the distal tip portion 263, and the threads 264 may in some respects comply with the ISO 80369-7 standard.

Similar to the catheter test apparatus 100, a piston 240 is disposed within a chamber 215 such that the piston 240 is longitudinally displaceable within the chamber 215 and the piston 240 is biased toward a distal end cap 220 via a biasing member 245 exerting a force of a predetermined magnitude on the piston 240. The elongate member 230 is coupled to the piston 240 and extends distally through the aperture 222. The elongate member 230 includes a gauge portion 235 at a distal end 231.

The body 210 may comprise an opening 218 in a side wall to provide for visible observation of the piston 240 within the chamber 215. The piston 240 may comprise a portion comprising a first color 243 (see FIG. 6) and a portion comprising a second color 244 (see FIG. 7), and the portion comprising the first color 243 may be disposed proximal of the portion comprising the second color 244. The opening 218 may be positioned on the body 210 so that, based on the longitudinal position of the piston 240 in the chamber 215, each of the first color 243 and the second color 244 may be visible through the opening 218. The first color 243 and the second color 244 may at least partially indicate to the medical practitioner the absence or presence, respectfully, of an obstruction 188 within the lumen 182 of the catheter 180.

The plunger rod 270 is coupled to the body 210 at a proximal end 212 so as to establish co-movement of the body 210 and the plunger rod 270. In some embodiments, the body 210 and plunger rod 270 may be integrally formed. The plunger rod 270 may comprise a flange 273 at a proximal end 272 so as to provide for the application of a distally directed force 274 on the plunger rod 270 by the thumb of the medical practitioner. The body 210 is longitudinally displaceable within the housing 260 between 1) a distal most position, wherein the distal end cap 220 of the body 210 is adjacent the distal end cap 265 of the housing 260 at which position the elongate member 230 is fully extended within the lumen 182 (see FIGS. 6) and 2) a proximal most position as shown in FIG. 5, wherein the elongate member 230 is fully retracted within an aperture 266.

FIG. 6 illustrates the catheter test apparatus 200 in operative engagement with the catheter 180 without an obstruction 188 present in the lumen 182. The body 210 is displaced distally so that the distal end cap 220 of the body 210 is disposed adjacent the distal end cap 265 of the housing 260. The gauge portion 235 of the elongate member 230 is distally displaced within the lumen 182 beyond the distal end of the catheter hub 184. The piston 240 is disposed adjacent the distal end cap 220 of the body 210 as exerted on by the biasing member 245 and the first color 243 is visible through the opening 218. The first color 243 may be a color generally regarded as positive, for example, the color green.

FIG. 7 illustrates the catheter test apparatus 200 in operative engagement with the catheter 180 with an obstruction 188 present in the lumen 182. The body 210 is displaced distally so that the gauge portion 235 engages the obstruction 188 preventing further insertion of the elongate member 230 within the lumen 182. The body 210 is distally displaced further, via a force applied to the plunger rod 270, such that the piston 240 is proximally displaced away from the distal end cap 220 in opposition to the biasing member 245, via a reaction force exerted on the elongate member 230 by the obstruction 188. As the piston 240 is disposed proximally away from distal end cap 220, a second color 252 is visible through the opening 218. The second color 244 may be a color generally regarded as negative, for example, the color red.

Use of the catheter test apparatus 200 described above to test a catheter for the presence of an obstruction may include any of the following steps or processes, each of which may be optional or interchanged. The following steps or processes may be performed in any order unless specifically defined. The following steps or processes may also be performed at a location other than the location of manufacture of the catheter. The following steps or processes may also be performed at the location of use of the catheter. The following steps or processes may also be performed while the catheter is inserted within the vasculature of a patient.

The medical practitioner may provide a catheter to be used in a vascular procedure which may include removing the catheter from a sterile package. The medical practitioner may provide a catheter test apparatus which may include removing the catheter from a sterile package. Providing the catheter test apparatus may include separating the sterile package containing the catheter test apparatus from a sterile package containing a vascular device to be inserted into the catheter to be tested without compromising the sterility of the vascular device and/or the catheter test apparatus. The medical practitioner may determine the suitability of the catheter test apparatus to test the catheter by comparing information contained in indicia disposed on the sterile package of the catheter test apparatus with one or more parameters of the catheter or vascular device.

The medical practitioner may threadably couple the catheter test apparatus to the hub of the catheter. The medical practitioner may apply a distally directed force on the plunger rod so as to distally displace the plunger rod and thereby insert the elongate member including the gauge portion at the distal end of the elongate member through the hub and into the lumen of the catheter until the body is fully displaced to the distal end of the housing or until a resistance to displacement of the plunger rod is felt by the medical practitioner. If a resistance to displacement is felt by the medical practitioner, the medical practitioner may apply an increasing magnitude of distal force to the plunger while observing the color of the piston displayed through an opening in the body to determine if further displacement may be achieved without causing the displayed color to change from a first color to a second color. The medical practitioner may 1) conclude that the catheter is obstruction free if only the first color is displayed during distal displacement of the plunger rod, or 2) conclude that the catheter contains an obstruction if the second color is displayed during distal displacement of the plunger rod.

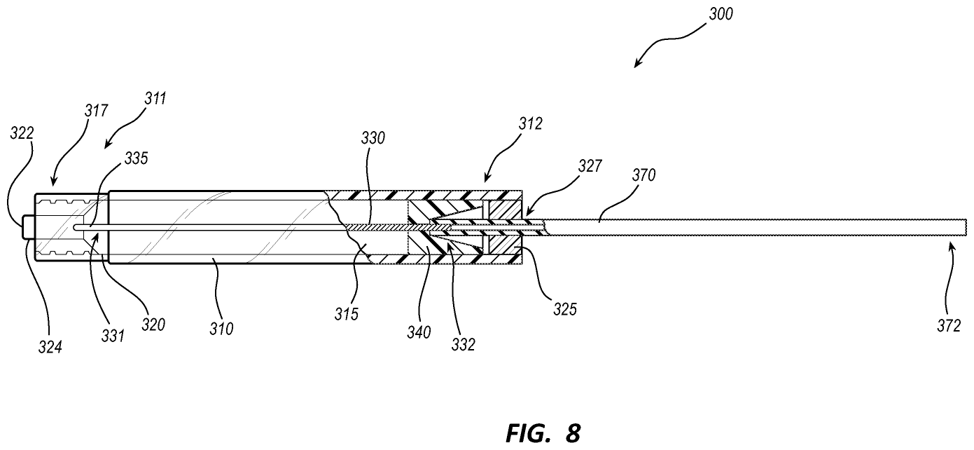

FIGS. 8, 9, and 10 illustrate another embodiment of a catheter test apparatus 300 that can, in certain respects, resemble components of the catheter test apparatus 100 in connection with FIGS. 1, 2, 3A, 3B, 4A, and 4B and/or catheter test apparatus 200 described in connection with FIGS. 5, 6 and 7. It will be appreciated that the illustrated embodiments may have analogous features. Accordingly, like features are designated with like reference numerals, with the leading digits incremented to “3.” For instance, the body is designated as “110” in FIGS. 1, 2, 3A, 3B, 4A, and 4B, and “210” in FIGS. 5, 6 and 7, and a body is designated as “310” in FIGS. 8, 9 and 10. Relevant disclosure set forth above regarding similarly identified features thus may not be repeated hereafter. Moreover, specific features of the catheter test apparatus 100, 200 and related components shown in FIGS. 1, 2, 3A, 3B, 4A, 4B, 5, 6, and 7 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the system of the catheter test apparatus 300 of FIGS. 8, 9 and 10. Any suitable combination of the features, and variations of the same, described with respect to the system of the catheter test apparatus 100, 200 and components illustrated in FIGS. 1, 2, 3A, 3B, 4A, 4B, 5, 6, and 7 can be employed with the system of the catheter test apparatus 300 and components of FIGS. 8, 9, and 10 and vice versa.

FIG. 8 is a side view of the catheter test apparatus 300. The catheter test apparatus 300 comprises a body 310 defining a chamber 315 extending between a distal end cap 320 and a proximal end cap 325. A piston 340 is disposed within the chamber 315. The piston 340 is coupled to a plunger rod 370 and an elongate member 330 establishing co-movement of the elongate member 330, the piston 340, and the plunger rod 370. The elongate member 330 extends distally away from the piston 340 and is displaceable through an aperture 322 in the distal end cap 320. The plunger rod 370 extends proximally away from the piston 340 and is disposed through an aperture 327 of the proximal end cap 325, and the plunger rod further extends proximally away from the proximal end of the housing 312. The elongate member 330 includes a gauge portion 335 at a distal end 331.

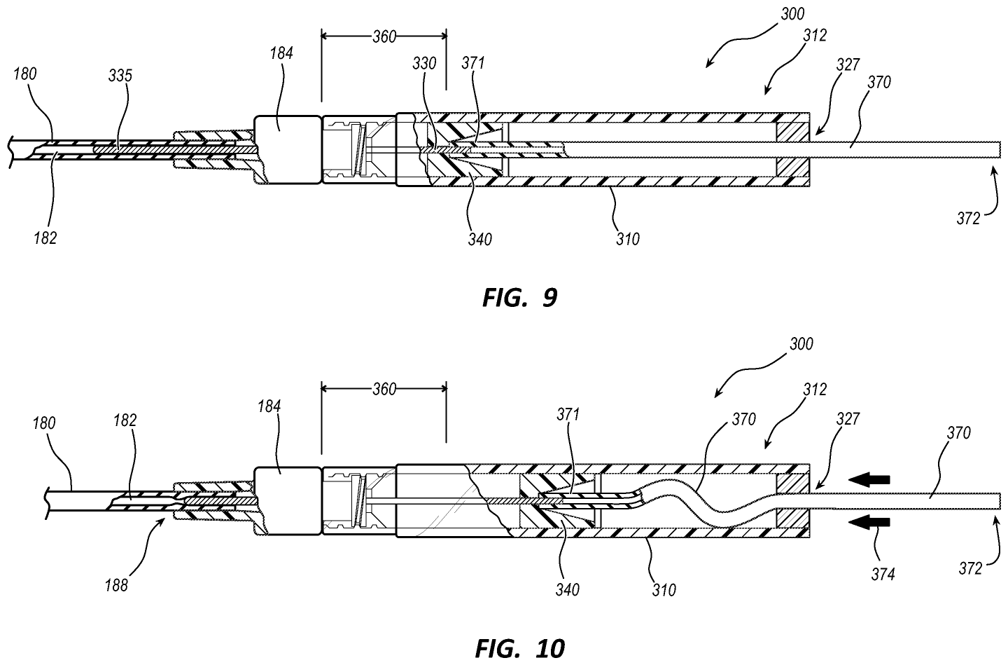

The distal end cap 320 may also include a distal tip portion 324, and the aperture 322 may also extend through the distal tip portion 324. The distal end cap 320 may also include threads 317 configured to engage threads of the catheter hub 184 (see FIGS. 9 and 10). The distal end cap 320, the distal tip portion 324, and the threads 317 may in some respects comply with the ISO 80369-7 standard.

The body 310 may be formed of a transparent or translucent material such that the piston 340 is visible within the chamber 315.

The plunger rod 370 is constructed to transfer a compressive force 374 to the piston 340. The plunger rod 370 may also be constructed to prevent transfer of the compressive force 374 (see FIG. 10) exceeding a predetermined magnitude as may be applied to a proximal end 372. In some embodiments, the plunger rod 370 may be constructed to buckle (experience column failure) if the compressive force 374 exceeds the predetermined magnitude. In some embodiments, the plunger rod 370 may comprise structural characteristics of an elongate vascular device. As such, the plunger rod 370 may buckle in a similar manner as an elongate portion of a catheter when exposed to a compressive force 374 exceeding the predetermined magnitude.

The length of the elongate member 330 may be defined by an anticipated location of an obstruction 188 along the length of the lumen 182. In some instances, insertion of the gauge portion 335 beyond the distal end of the catheter hub 184 may provide the assurance desired by the medical practitioner. As such, the length of the elongate member 330 may be at least partially defined by the distance between the distal end of the catheter hub 184 and the distal end of the piston 340 when a distal end 311 of the body 310 is in operative engagement with the catheter hub 184 of the catheter 180. In the illustrated embodiment, the length of the elongate member 330 is longer than the distance between the distal end of the catheter hub 184 and the distal end of the piston 340 when the distal end 311 of the body 310 is in operative engagement with the catheter hub 184 of the catheter 180. The length of the elongate member 330 together with the distance between the distal end of the catheter hub 184 and the distal end of the piston 340 when the distal end 311 of the body 310 is in operative engagement with the catheter hub 184 may at least partially define a predetermined distance 360. In some embodiments, the body 310 may include a line or other marking to provide visual indication of the predetermined distance 360.

FIG. 9 illustrates the catheter test apparatus 300 in operative engagement with the catheter 180 without an obstruction 188 present in the lumen 182. The gauge portion 335 of the elongate member 330 is distally displaced within the lumen 182 beyond the distal end of the catheter hub 184. The piston 340 is displaced distally in response to a compressive force 374 applied to the plunger rod 370 so that the piston 340 is disposed adjacent the distal end cap 320, which is also distal of the predetermined distance 360 providing an indication to the medical practitioner of the absence of an obstruction 188.

FIG. 10 illustrates the catheter test apparatus 300 in operative engagement with the catheter 180 with an obstruction 188 present in the lumen 182. The plunger rod 370 is displaced distally by the application of a compressive force 374 until the gauge portion 335 engages the obstruction 188 preventing further insertion of the elongate member 330 within the lumen 182 at which point the plunger rod 370 buckles. The piston 340 is disposed proximal of the predetermined distance 360 providing an indication to the medical practitioner of the presence of the obstruction 188.

Use of the catheter test apparatus 300 described above to test a catheter for the presence of an obstruction may include any of the following steps or processes, each of which may be optional or interchanged. The following steps or processes may be performed in any order unless specifically defined. The following steps or processes may also be performed at a location other than the location of manufacture of the catheter. The following steps or processes may also be performed at the location of use of the catheter. The following steps or processes may also be performed while the catheter is inserted within the vasculature of a patient.

The medical practitioner may provide a catheter to be used in a vascular procedure which may include removing the catheter from a sterile package. The medical practitioner may provide a catheter test apparatus which may include removing the catheter from a sterile package. Providing the catheter test apparatus may include separating the sterile package containing the catheter test apparatus from a sterile package containing a vascular device to be inserted into the catheter to be tested without compromising the sterility of the vascular device and/or the catheter test apparatus. The medical practitioner may determine the suitability of the catheter test apparatus to test the catheter by comparing information contained in indicia disposed on the sterile package of the catheter test apparatus with one or more parameters of the catheter or vascular device.

The medical practitioner may threadably couple the catheter test apparatus to the hub of the catheter. The medical practitioner may distally displace the plunger rod and thereby insert the elongate member including the gauge portion at the distal end of the elongate member through the hub and into the lumen of the catheter by the application of a distally directed force to the plunger rod. In some embodiments, the force may be applied to the proximal end of the plunger rod. The medical practitioner may apply an increasing magnitude of distal directed force to the plunger rod so as to overcome any insertion resistance until the plunger rod buckles. The medical practitioner may observe the position of the piston which position may also be consistent with the position of the proximal end of the elongate member to determine if the proximal end of the elongate member is distal or proximal of a predetermined distance from the catheter hub. The medical practitioner may 1) conclude that the catheter is obstruction free if the proximal end of the elongate member is distal of the predetermined distance, or 2) conclude that the catheter contains an obstruction if the proximal end of the elongate member is proximal of the predetermined distance.

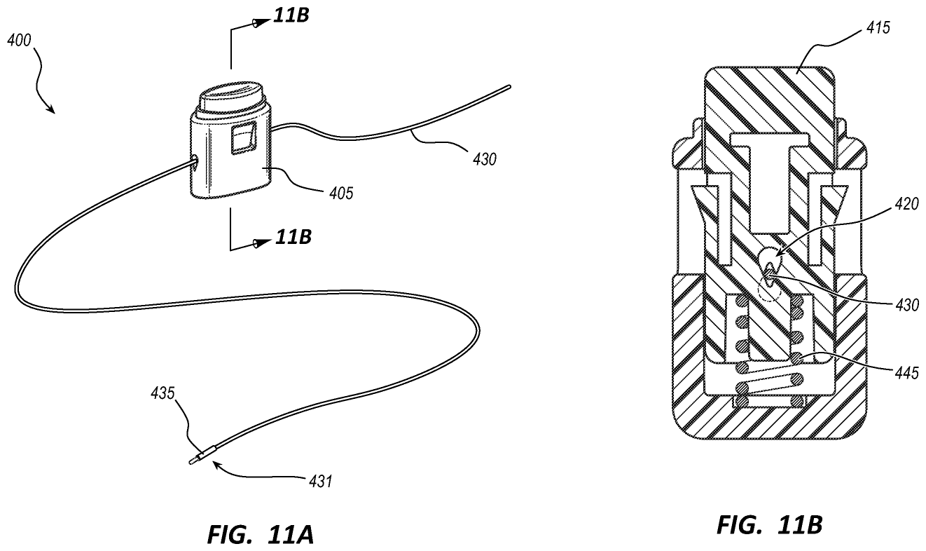

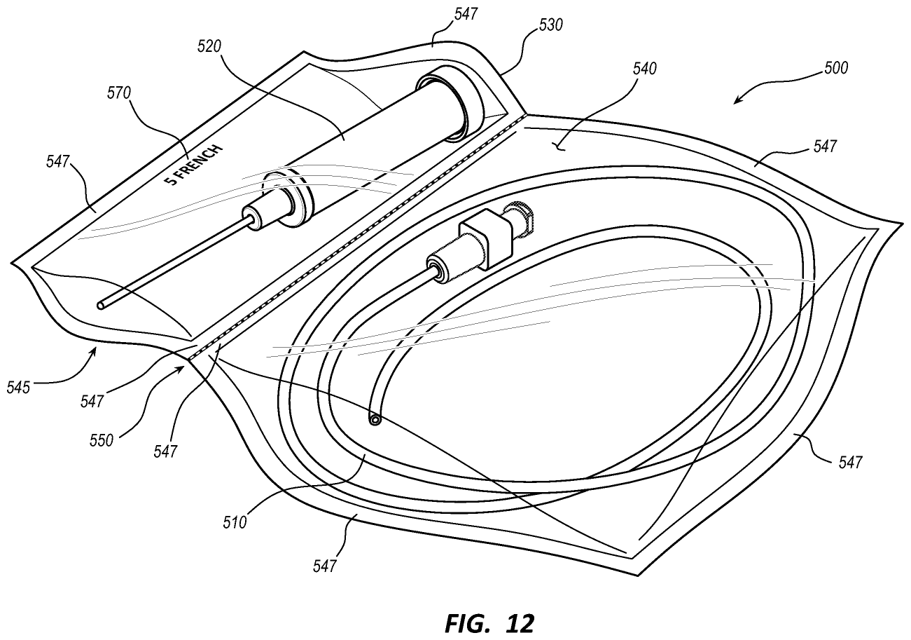

FIGS. 11A and 11B illustrate another embodiment of a catheter test apparatus 400. In some instances, the distal end of a catheter may have an increased propensity for an obstruction or defect. In other instances, catheter characteristics in combination with a vascular pathway may cause a kink in the catheter. Hence, in such instances, a medical practitioner may desire to verify the lumen integrity of a catheter along the entire length of the catheter or a significant portion of the length. The catheter test apparatus 400 comprises a flexible elongate member 430 which may in some embodiments resemble a guidewire. The elongate member 430 includes a gauge portion 435 at a distal end 431. The elongate member 430 may be sized to extend the entire length of the catheter to be tested and, therefore, displace the gauge portion 435 along the entire length of catheter. As such, in an instance where the catheter does not contain an obstruction, the gauge portion 435 may pass through the entire lumen of the catheter thus providing the medical practitioner with an indication of the absence of an obstruction. Conversely, in an instance where the catheter does contain an obstruction, the gauge portion 435 may pass through the lumen to the location of the obstruction or kink and then may be prevented from passing further through the lumen thus providing the medical practitioner with an indication of the presence of an obstruction.

In the illustrated embodiment, the catheter test apparatus 400 may include a clamp 405. The clamp 405 may be configured to selectively attach to the elongate member 430 so as to prevent displacement along the elongate member 430 unless a longitudinal force of the elongate member 430 exceeds a predetermined magnitude. In other words, the clamp 405 may be configured to allow displacement of the elongate member 430 relative to the clamp 405 if the longitudinal force of the elongate member 430 is above the predetermined magnitude and prevent displacement along the elongate member 430 relative to the clamp 405 if the longitudinal force of the elongate member 430 is equal to or less than the predetermined magnitude.

The clamp 405 may include a button 415 to selectively enable free displacement of the elongate member 430 relative to the clamp 405. In other words, the clamp 405 may be constructed so that a medical practitioner may 1) upon pressing the button 415, freely dispose the clamp 405 at any position along the length of the elongate member 430, and 2) upon releasing the button 415, secure the clamp 405 to the elongate member 430. The clamp 405 may include a clamping region 420 through which the elongate member 430 may be inserted such that displacement of the clamp 405 along the elongate member 430 coincides with displacement of the elongate member 430 through the clamping region 420.

The clamp 405 may include a biasing member 445. In some instances, the magnitude of longitudinal force that is applied to the elongate member 430 via the clamp 450 may be defined by the biasing member 445. In other words, the biasing member 445 may produce a clamping force on the elongate member 430 when the elongate member 430 is disposed through the clamping region 420 so as to establish the predetermined magnitude of longitudinal force. In the illustrated embodiment, pressing the button 415 may oppose the biasing member 445 and remove the clamping force.

In some instances, if the gauge portion 435 meets an obstruction, the force exerted by the obstruction may overcome the clamping force of the biasing member 445 causing the elongate member 430 to slip with respect to the clamp 405. Thus, the clamping force provided by the biasing member 445 may be configured to facilitate testing of the diameter of an entire catheter by advancing the gauge portion 534 along the catheter. If the elongate member 430 does not slip with respect to the clamp 405, no obstruction preventing passage of the gauge portion 435 may be present, while slipping may be understood as indicating an obstruction. As with the catheter test device 100 of FIG. 1, in some embodiments the gauge portion 435 of the embodiment of FIG. 11A may be the same diameter as the rest of the elongate member 430. In other words, the elongate member 430 may have a constant outside diameter along its length, including the gauge portion 435.

In certain embodiments, the clamping region 420, button 415, and biasing member 445 may all be disposed on a shuttle with respect to the body of the clamp 405. A longitudinal biasing member may tend to maintain the longitudinal position of the shuttle with respect to the body of the clamp 405 until an obstruction is encountered during testing. Meeting an obstruction that overcomes the maintenance force of the longitudinal biasing member may cause the shuttle to move with respect to the body of the clamp 405. This movement may expose a colored portion and/or may extend a feature from the body of the clamp 405 to provide an indicia that an obstruction was encountered. In other words, in some embodiments, the embodiment of FIG. 11A may be configured such that an element analogous to the plunger 140 of FIG. 2 extends from the body of the clamp 405 when an obstruction is encountered. Thus, in some embodiments, the biasing member 445 may be configured to provide a greater clamping force on the elongate member 430 such that the elongate member 430 is not configured to slip in the event an obstruction is encountered during testing.

Use of the catheter test apparatus 400 described above may include any of the following steps or processes, each of which may be optional or interchanged. The following steps or processes may be performed in any order unless specifically defined. The following steps or processes may also be performed at a location other than the location of manufacture of the catheter. The following steps or processes may also be performed at the location of use of the catheter. The following steps or processes may also be performed while the catheter is inserted within the vasculature of a patient.

The medical practitioner may provide a catheter to be used in a vascular procedure which may include removing the catheter from a sterile package. The medical practitioner may provide a catheter test apparatus which may include removing the catheter from a sterile package. Providing the catheter test apparatus may include separating the sterile package containing the catheter test apparatus from a sterile package containing a vascular device to be inserted into the catheter to be tested without compromising the sterility of the vascular device and/or the catheter test apparatus. The medical practitioner may determine the suitability of the catheter test apparatus to test the catheter by comparing information contained in indicia disposed on the sterile package of the catheter test apparatus with one or more parameters of the catheter.

In use, the medical practitioner may insert the distal end of the elongate member, including the gauge portion at the distal end, through the hub of a catheter and into the lumen. The medical practitioner may dispose the clamp on the elongate member at a position proximal of the hub and apply a distal force to the clamp to exert an insertion force on the elongate member. During insertion, the medical practitioner may repeatedly reposition the clamp on the elongate member. During insertion, the longitudinal force on the elongate member as applied by the clamp may overcome insertion resistance. The medical practitioner may continue the insertion process until the gauge portion exits the distal end of the catheter lumen or until the gauge portion passes a location of the catheter lumen as predetermined by the medical practitioner at which point the medical practitioner may conclude that the catheter is obstruction free. The medical practitioner may remove the elongate member from the catheter lumen and proceed with insertion of a vascular device into the lumen of the catheter.

During insertion, if an insertion resistance causes the clamp to displace relative to the elongate member, i.e., overcome the predetermined magnitude, when the distal force is applied to the clamp, the medical practitioner may conclude that an obstruction is present in the lumen. As noted above, other indicia of obstructions may include colors or the extension of other elements. For example, in some embodiments, an obstruction force may cause a shuttle to move within the body of the clamp 405 to extend an element to indicate an obstruction has been reached.

The medical practitioner may remove the elongate member from the catheter lumen and insert the elongate member into a different catheter.

FIG. 12 illustrates a kit 500 including a catheter test apparatus 520 and a vascular device 510. The catheter test apparatus 520 may be any one of the catheter test apparatus 100, 200, 300 or 400 described above. The vascular device 510 may be any one of a catheter, a guidewire, or any other vascular device suitable for insertion within a catheter lumen. The kit 500 may also include a package 530 which may comprise at least two sterile compartments 535 each containing one of the catheter test apparatus 520 and the vascular device 510. The package 530 may provide a sterile barrier to maintain sterility of the catheter test apparatus 520 and vascular device 510.

The package 530 may be formed opposing packaging films 540, 545 coupled together along perimeter 547 surrounding the at least two sterile compartments 535. One or more of the packaging films 540, 545 may be flexible or semi-flexible. One or more of the packaging films 540, 545 may also be transparent or translucent. One or more of the packaging films 540, 545 may also be breathable or comprise a breathable portion.

The at least two sterile compartments 535 may be coupled to each other along a portion of weakened integrity, for example, a perforation, such that the at least two sterile compartments 535 may be separated without 1) compromising the sterility of at least the vascular device 510 or 2) compromising the sterility of either the vascular device 520 or the catheter test apparatus 520.

The package 530 may include indicia 570 pertaining to one or more parameters of the catheter test apparatus 520, including one or more parameters of a gauge portion of the catheter test apparatus 520. The indicia 570 may indicate the suitability of the catheter test apparatus 520 for testing a catheter.

Changes may be made to the details of the above-described embodiments without departing from the underlying principles of the invention. The scope of the present invention should, therefore, be determined only by the following claims.

1. A catheter test apparatus, comprising:
a body comprising:
a chamber extending at least partially through the body, and
a distal end cap including an aperture extending therethrough;
a piston disposed within the chamber; and
an elongate member configured to be disposed within a lumen of a catheter, wherein the elongate member is disposed through the aperture and is coupled to the piston such that longitudinal displacement of the elongate member correlates to displacement of the piston within the chamber,
wherein the elongate member comprises a gauge portion disposed at a distal end of the elongate member, the gauge portion sized to be 1) displaceable within the lumen if a minimum inside diameter of the lumen is greater than a predetermined limit and 2) not displaceable within the lumen if the minimum inside diameter is below the predetermined limit; and
wherein the catheter test apparatus is configured to provide an indication to a medical practitioner of a presence and/or an absence of an obstruction within the lumen such that, in use, 1) a position of the piston proximal of a predetermined distance from a hub of the catheter indicates the presence of the obstruction within the lumen, and 2) a position of the piston adjacent to or distal of a predetermined distance from the hub indicates the absence of the obstruction within the lumen.

2. The apparatus of claim 1, further comprising a plunger rod coupled to the piston and extending proximally beyond a proximal end of the body, the plunger rod configured to transfer a compressive force manually applied to plunger rod to the piston,
wherein the plunger rod is structured to buckle if the force applied to the elongate member exceeds a predetermined magnitude.

3. The apparatus of claim 1, further comprising a biasing member disposed between the body and the piston, the biasing member configured to apply a predetermined magnitude of force to the piston toward a distal end of the chamber such that the piston is displaceable away from the distal end when a force exceeding the predetermined magnitude is proximally applied to the elongate member.

4. The apparatus of claim 3, wherein in use, the presence of an obstruction causes the elongate member to displace proximally in opposition to the predetermined magnitude of force.

5. The apparatus of claim 4, further comprising a housing configured to at least partially encapsulate the body, the housing comprising:
a cavity extending longitudinally at least partially through the housing; and
an end cap disposed at a distal end of the housing, the end cap including a housing aperture extending therethrough,
wherein the body is disposed within the cavity and the elongate member is disposed within the housing aperture.

6. The apparatus of claim 5, further comprising plunger rod coupled to the body establishing co-movement of the plunger rod and the body, wherein the plunger rod extends proximally beyond a proximal end of the cavity.

7. The apparatus of claim 5, wherein the body comprises an opening in a side wall of the body such that the piston is visible through the opening.

8. The apparatus of claim 7, wherein the piston comprises a first colored portion comprising a first color and a second colored portion disposed distal of the first colored portion, the second colored portion comprising a second color, and
wherein when the piston is disposed adjacent to or distal of the predetermined distance, the first color is visible through the opening, and when the piston is disposed proximal of the predetermined distance, the second color is visible through the opening.

9. The apparatus of claim 1, wherein the elongate member comprises a rigid portion and a flexible portion.

10. The apparatus of claim 1, wherein the elongate member is sized to be inserted within the lumen at least beyond a distal end of the catheter.

11. A method of testing a catheter, comprising:
providing a catheter test apparatus comprising an elongate member comprising a gauge portion disposed at a distal end of the elongate member, the gauge portion sized to be 1) displaceable within a lumen of a catheter if a minimum inside diameter of the lumen is greater than a predetermined limit and 2) not displaceable within the lumen if the minimum inside diameter is below the predetermined limit,
wherein the catheter test apparatus is configured to provide an indication to a medical practitioner of a presence and/or an absence of an obstruction within the lumen such that, in use, 1) a position of a proximal end of the elongate member proximal of a predetermined distance from a hub of the catheter indicates the presence of an obstruction within the lumen, and 2) a position of the proximal end adjacent to or distal of a predetermined distance from the hub indicates the absence of the obstruction within the lumen;
providing a catheter to be tested;
inserting the gauge portion through a hub of the catheter and disposing the gauge portion adjacent a proximal opening of a lumen of the catheter;
applying a force up to a predetermined magnitude distally to the elongate member;
allowing the gauge portion to displace distally in response to the force;
visually assessing the position of the proximal end of the elongate member with respect to the predetermined distance; and
determining if the position of the proximal end of the elongate member is proximal of the predetermined distance.

12. The method of claim 11, wherein the method is performed at a location of use of the catheter.

13. The method of claim 11, further comprising removing the catheter test apparatus from a sterile package.

14. The method of claim 11, further comprising removing the catheter from a sterile package.

15. A kit for testing a catheter comprising:
a catheter test apparatus comprising an elongate member including a gauge portion disposed at a distal end of the elongate member, the gauge portion sized to be 1) displaceable within a lumen of a catheter if a minimum inside diameter of the lumen is greater than a predetermined limit and 2) not displaceable within the lumen if the minimum inside diameter is below the predetermined limit,
wherein the catheter test apparatus is configured to provide an indication to a medical practitioner of a presence and/or an absence of an obstruction within the lumen such that, in use, 1) a position of the piston proximal of a predetermined distance from a hub of the catheter indicates the presence of an obstruction within the lumen, and 2) a position of the piston adjacent to or distal of a predetermined distance from the hub indicates the absence of the obstruction within the lumen; and
a vascular device to be inserted within a lumen of a catheter to be tested; and
a package configured to contain the catheter test apparatus and the vascular medical device, the package comprising at least two sterile compartments,
wherein each of the catheter test apparatus and the vascular device is disposed individually within one of the at least two sterile compartments.

16. The kit of claim 15, wherein each of the at least two sterile compartments is formed of opposing packaging films coupled together along a perimeter surrounding the sterile compartment.

17. The kit of claim 16, wherein the at least two sterile compartments are formed from at least one common packaging film such that the at least two sterile compartments are coupled together via the at least one common packaging film.

18. The kit of claim 17, wherein the at least one common packaging film comprises a portion of weakened integrity disposed between the at least two sterile compartments such that the at least two sterile compartments may be separated from each other without compromising a sterile barrier of at least one of the at least two sterile compartments.

19. The kit of claim 15, wherein at least one of the at least two sterile compartments comprises indicia, and
wherein the indicia contains information pertaining to at least one parameter of the catheter test apparatus.

20. The kit of claim 19, wherein the indicia contains information pertaining to the at least one parameter of the gauge portion.
